(19) United States
(12) Patent Application Publication
(10) Pub. No.: US 2026/0345248 A1
(43) Pub. Date: Nov. 5, 2026

(54) METHOD OF PREPARING A SUBJECT FOR A MEDICAL INTERVENTION USING A MAGNETIC-FIELD BASED SYSTEM

(71) Applicant: STARPAX BIOPHARMA INC., Montréal (CA)

(72) Inventors: Charles TREMBLAY, Ville Saint-Laurent (CA); Sylvain MARTEL, Île Bizard (CA); Michel GAREAU, Bromont (CA); Thierry PAGÉ, Montréal (CA); Dumitru LOGHIN, Laval (CA); Maxime LATULIPPE, Montréal (CA)

(21) Appl. No.: 19/101,797

(22) PCT Filed: Aug. 24, 2023
(86) PCT No.: PCT/CA2023/051118

§ 371 Date: Feb. 6, 2025

Related U.S. Application Data
(60) Provisional application No. 63/373,452, filed on Aug. 24, 2022.
(60) Provisional application No. 63/384,182, filed on Nov. 17, 2022.

(51) Int. Cl. A61B 34/20; A61B 5/055; A61B 90/00

(52) U.S. Cl. CPC A61B 34/20; A61B 5/055; A61B 90/39; A61B 2034/2051; A61B 2090/395

(57) ABSTRACT

A method for performing a medical intervention on a subject using magneto-responsive entities; it includes identifying reference points with respect to the subject indicative of a location of a target zone in the subject for one or more of treatment, diagnosis and imaging; using the reference points to position the subject on a resting device in proximity to one or more sources for generating the magnetic field, the positioning performed in relation to the one or more sources; performing imagery of the subject after the subject is positioned on the table to confirm the location of the target zone in the subject; whereby the magneto-responsive entities are to be introduced into the subject positioned on the table, and the magnetic field is applied to the magneto-responsive entities to obtain a response from the magneto-responsive entities.

The present application claims priority from U.S. provisional patent application No. 63/384,182, filed on Nov. 17, 2022, incorporated herein by reference, and U.S. provisional patent application No. 63/373,452, filed on Aug. 24, 2022, incorporated herein by reference.

### TECHNICAL FIELD

The present disclosure relates to targeted intervention in a subject using a magnetic field, and more particularly to targeted intervention in a subject using magneto-responsive entities that are steered with a magnetic field.

### BACKGROUND

Use of a magnetic field for medical interventions is becoming more prevalent. For instance, the following patent documents describe prior art medical applications harnessing a magnetic field: U.S. Pat. Nos. 9,655,539, 9,381,063, 9,220,425, 8,986,214, 8,684,010, 8,457,714, US20130006100, US20120310111, US20120289822, US20120288838, US 20110092808, U.S. Pat. No. 7,873,401, US 20100305402, US 20090275828, US20090248014, US20050096589, etc.

Recently, the use of magneto-responsive entities, used with an applied magnetic field, has also been studied for medical purposes.

Magneto-responsive entities are defined as untethered entities where the source of propulsion or the system responsible for the displacement of the entity is part of, attached to, or embedded in the entity itself. Magneto-responsive entities include a group of objects or microorganisms and any biological system or hybrid system including micro-and nano-systems or structures made of biological and/or synthetic (including chemical, artificial, etc.) materials and/or components where the directional motion can be influenced by inducing a torque from a directional magnetic field (e.g. from a permanent magnet) or electro-magnetic field (magnetic field includes here electro-magnetic field generated by an electrical current flowing in a conductor), a method referred to here as magnetotaxis where the direction of motion of such magneto-responsive entities is influenced by a directional magnetic field (the magneto-responsive entities can also be functionalized and be attached to other structures if required). Examples of such magneto-responsive entities include but are not limited to a single or a group (swarm, agglomeration, aggregate, etc.) of flagellated Magnetotactic Bacteria (MTB), or other bacteria or other microorganisms capable of self-propulsion and influenced for the purpose of directional control by a directional magnetic field that could have been modified previously accordingly from various methods including but not limited to cultivation parameters, genetics, or attached, embedded to other entities modified to allow control by magnetotaxis such as other cells (including red blood cells), or attached to a synthetic structure that can be influenced by a directional magnetic field or gradient, or by adding micro- or nano-components to the bacteria, cells, or other microorganisms to make the directional motion of the implementation including hybrid (made of biological and synthetic components) implementation sensitive to magnetotaxis or a directional magnetic field such as the one capable of influencing the direction of a magnetic nano-compass needle. The magneto-responsive entities may be north-seeking, south-seeking or a mixture of both.

U.S. Pat. No. 9,905,347, incorporated herein by reference, describes a system for steering magneto-responsive entities in a subject. U.S. Pat. No. 9,905,347 describes a system and method for generating a 3D-convergence point using at least three sets of magnetic field sources arranged along three axes or in three planes.

Such a magnetotactic system requires a high degree of precision in order to improve the target intervention using the magneto-responsive entities.

### SUMMARY

The present disclosure relates to a method for positioning a subject for a medical intervention using a magnetotatic system. Prior to the medical intervention, the method involves the use of reference points on the subject (e.g. fiduciary markers) to identify the target zone for the magnetotactic bacteria using the magnetotactic system. Imagery is then used to reconfirm the position of the target zone prior to the medical intervention.

A broad aspect is a method for performing a medical intervention on a subject using magneto-responsive entities, tethered to at least one of a treatment agent, an imaging agent and a diagnostic agent, that responds to a magnetic field, the magneto-responsive entities steered in a body of the subject using the magnetic field, the magneto-responsive entities delivering the at least one of the treatment agent, the imaging agent and the diagnostic agent in the subject. The method includes identifying reference points with respect to the subject indicative of a location of a target zone in the subject for one or more of treatment, diagnosis and imaging; using the reference points to position the subject on a resting device in proximity to one or more sources for generating the magnetic field, the positioning performed in relation to the one or more sources; performing imagery of the subject after the subject is positioned on the table to confirm the location of the target zone in the subject; whereby the magneto-responsive entities are to be introduced into the subject positioned on the table, and the magnetic field is applied to the magneto-responsive entities to obtain a response from the magneto-responsive entities using the magnetic field generated by the proximate one or more sources to direct the magneto-responsive entities to the target zone aided by the reference points after the location of the target zone has been confirmed.

In some embodiments, the target zone may be immobilized during the conducting of the medical intervention.

In some embodiments, the performing imagery may be used to confirm the position of the reference points with relation to the target zone.

In some embodiments, the method may include, prior to the positioning of the subject on the table, positioning the subject on a reference table to confirm the reference points with respect to the reference table.

In some embodiments, the imagery may be performed using a C-arm.

In some embodiments, the imagery may be performed using ultrasound coupled to infrared positioning magnetic sensors.

In some embodiments, the introducing may be performed via injection using ultrasound to assist in positioning of a needle used for the injection.

In some embodiments, the reference points may include tattoos applied to the subject.

In some embodiments, the reference points may include one or more of fiducial markers, landmarks on the body of the subject, and the target zone.

In some embodiments, the imagery may be performed to calculate an offset from the location of the target zone and the position of the subject on the table.

In some embodiments, the target zone may define a tumor.

In some embodiments, the target zone may correspond to a hypoxic zone.

In some embodiments, the target zone may be a necrotic zone.

In some embodiments, the non-vascularized hypoxic zone may be associated to an ischemic stroke, pulmonary hypertension, ischemic cardiopathy or diabetic retinopathy.

In some embodiments, the one or more sources may include three pairs of magnetic heads, wherein each pair may be aligned with regard to a distinct one of three axes including a x axis, a y axis and a z-axis.

In some embodiments, the imagery may be performed after the injecting.

In some embodiments, the imagery may be performed before the injecting.

In some embodiments, the method may include calculating an isocenter shift between an expected position of the target zone, and a validated position of the target zone identified using imagery, wherein the positioning of the subject on the resting device is performed further using the isocenter shift.

Another broad aspect is a method for performing a medical intervention on a subject using magneto-responsive entities, tethered to at least one of a treatment agent, an imaging agent and a diagnostic agent, that responds to a magnetic field, the magneto-responsive entities steered in a body of the subject using the magnetic field, the magneto-responsive entities delivering the at least one of the treatment agent, the imaging agent and the diagnostic agent in the subject. The method includes defining reference points with respect to the subject indicative of a location of a target zone in the subject for one or more of treatment, diagnosis and imaging; using the reference points to position the subject on a resting device in proximity to one or more sources for generating the magnetic field, the positioning performed in relation to the one or more sources; performing imagery of the subject after the subject is positioned on the table to confirm an injection site; whereby the magneto-responsive entities are to be introduced into the subject positioned on the table, and the magnetic field is applied to the magneto-responsive entities to obtain a response from the magneto-responsive entities using the magnetic field generated by the one or more sources to direct the magneto-responsive entities to the target zone.

In some embodiments, the target zone may be immobilized during the conducting of the medical intervention.

In some embodiments, the target zone may be immobilized using immobilizing equipment receiving the subject, and further comprising marking a position of the immobilized subject with respect to the immobilizing equipment.

In some embodiments, the method may include defining initial reference points with respect to the subject for purposes of performing initial imagery of the subject, wherein the initial imagery identifies a location of the target zone; and calculating an isoshift from an isocenter defined by the initial reference points and the location of the target zone, wherein the reference points are defined using the isoshift.

In some embodiments, the initial imagery may include performing magnetic resonance imaging of the subject.

In some embodiments, the initial imagery may be registered with respect to the imagery of the subject after the subject is positioned on the table.

In some embodiments, the imagery may be performed using a C-arm performing cone-beam computed tomography.

In some embodiments, the using the reference points to position the subject may be performed with a laser system for positioning the reference points with respect to the one or more sources

In some embodiments, the introducing may be performed via injection using ultrasound to assist in positioning of a needle used for the injection.

In some embodiments, the reference points may include tattoos applied to the subject.

In some embodiments, the reference points may include one or more of fiducial markers, landmarks on the body of the subject, and the target zone.

In some embodiments, the target zone may define a tumor.

In some embodiments, the target zone may correspond to a hypoxic zone.

In some embodiments, the target zone may be a necrotic zone.

In some embodiments, the non-vascularized hypoxic zone may be associated to an ischemic stroke, pulmonary hypertension, ischemic cardiopathy or diabetic retinopathy.

In some embodiments, the one or more sources may include three pairs of magnetic heads, wherein each pair is aligned with regard to a distinct one of three axes including a x axis, a y axis and a z-axis.

In some embodiments, the imagery may be performed after the injecting.

In some embodiments, the imagery may be performed before the injecting.

In some embodiments, the method may include applying markers.

Another broad aspect is a method for preparing a subject for a medical intervention on a subject using magneto-responsive entities, tethered to at least one of a treatment agent, an imaging agent and a diagnostic agent, that responds to a magnetic field generated by a plurality of magnetic field sources, the magneto-responsive entities steered in a body of the subject using the magnetic field, the magneto-responsive entities delivering the at least one of the treatment agent, the imaging agent and the diagnostic agent in the subject. The method includes adjusting a position of the subject with respect to the isocenter of the plurality of magnetic field sources to at least improve alignment of a target zone of the subject with respect to the isocenter, the adjustment guided using reference points defined with respect to a body of the subject, the reference points being defined with respect to a body of the subject following an application of an isocenter shift calculated from a position of the target zone identified using imagery of the subject.

Another broad aspect is a method for preparing a subject for a medical intervention using magneto-responsive entities, tethered to at least one of a treatment agent, an imaging agent and a diagnostic agent, that responds to a magnetic field, the magneto-responsive entities steered in a body of the subject using the magnetic field, the magneto-responsive entities delivering the at least one of the treatment agent, the imaging agent and the diagnostic agent in the subject. The method includes defining reference points with respect to the subject indicative of a location of a target zone in the subject for one or more of treatment, diagnosis and imaging; using the reference points to position the subject on a resting device in proximity to one or more sources for generating the magnetic field, the positioning performed in relation to the one or more sources; performing imagery of the subject after the subject is positioned on the resting device; and confirming an injection site; whereby the magneto-responsive entities are to be introduced into the subject positioned on the table, and the magnetic field is applied to the magneto-responsive entities to obtain a response from the magneto-responsive entities using the magnetic field generated by the one or more sources to direct the magneto-responsive entities to the target zone.

### BRIEF DESCRIPTION OF THE DRAWINGS

The invention will be better understood by way of the following detailed description of embodiments of the invention with reference to the appended drawings, in which:

FIG. 1 is a drawing of a perspective view of an exemplary system for steering magneto-responsive entities in accordance with the disclosed embodiments;

FIG. 2 is a drawing of a perspective view of the system of FIG. 1, without the table, showing the magnetic head that may be positioned under the table;

FIG. 3 is a drawing of a perspective view of the system of FIG. 1, where a subject is laid on the table with their legs positioned for a medical intervention in the pelvis (such as rectal cancer, prostate cancer, interventions in the genital organs, etc.), and where a doctor is positioned under the exemplary support arch of the system, treating the subject;

FIG. 4 is a drawing of a top-down view of the system of FIG. 1 without the imaging device;

FIG. 5 is a drawing of a close-up of the magnetic head of the system of FIG. 1 that is to be located under the table of the system of FIG. 1;

FIG. 6 is a block diagram of an exemplary system for steering magneto-responsive entities;

FIG. 7 is an electric schematic of electrical components of an exemplary system for steering magneto-responsive entities in a subject;

FIG. 8 is a schematic of exemplary positions of the magnetic heads with respect to one another;

FIG. 9 is a drawing of a subject lying on a surgical table to register the position of the subject with respect to immobilizing equipment positioned on the surgical table;

FIG. 10 is a drawing of a subject lying on a surgical table with added fiducial markers;

FIG. 11 is a drawing of the addition of imaging markers to a subject for calculating an isocenter-shift;

FIG. 12 is a drawing of a subject undergoing imagery to position reference markers associated with the target zone;

FIG. 13 is a drawing of a subject being repositioned on the immobilizing equipment;

FIG. 14 is a drawing illustrating the calculation of a difference between the isocenter and the target zone, determined per the reference points added to the subject;

FIG. 15 is a drawing with the revised reference points for the target zone;

FIG. 16 is a drawing illustrating the repositioning of the resting device with the subject with respect to the magnetic coils of the magnetotactic system;

FIG. 17 is a drawing of a needle being advanced to, or in proximity of, the target zone;

FIG. 18 is a drawing of a C-arm being positioned around the subject for taking images to confirm the position of the needle and other subject reference points;

FIG. 19 is a drawing illustrating the injection of the magneto-responsive entities injected into the subject;

FIG. 20 is a drawing illustrating treatment of the subject using the magnetetotaxis system; and

FIG. 21 is a flowchart diagram of an exemplary method of preparing a subject for a medical procedure involving a magnetotactic system.

### DETAILED DESCRIPTION

The present disclosure relates to methods of positioning a subject for the purpose of performing a medical intervention using a magnetotactic system. As a high level of accuracy is required to guide the magneto-responsive entities to the target zone, the positioning of the subject with respect to the system, the positioning performed using the presence of external reference points on the subject, increases the accuracy of the targeting using the magneto-responsive entities.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

### DEFINITIONS

In the present disclosure, by “medical intervention,” it a meant a procedure performed on a subject for at least one of treatment (e.g., removal of a mass—tumor—through surgery), imaging (e.g., endoscopy, colonoscopy, optical coherence tomography, etc.) and diagnosis (which can involve one or more imaging techniques).

In the present disclosure, by “subject”, it is meant mammals and non-mammals. Mammals mean any member of the mammalia class including, but not limited to, humans. Non-mammals include birds, reptiles, etc. The term “subject” should not bring on any limitations as to the sex or age. Even though the configuration of the system has been determined to accommodate a human subject adopting different intervention positions as explained herein, it will be understood that the present system can be used with non-human subjects (i.e. animals), without departing from the present teachings, provided that the animal can fit within the space defined by the structure of the system and there is sufficient room for the members of the medical staff (e.g. veterinarian; technician) to access the subject and/or circulate around the subject.

In the present disclosure, by “target zone”, it is meant an area in the space (e.g. in 3D space) in which, or towards which, the magneto-responsive entities are to aggregate, following the navigation of the magneto-responsive entities using a magnetic field generated by the magnetotactic system. The 3D convergence point may be located in the target zone. An exemplary target zone may be a portion of a tumor, e.g., a portion of the tumor with one or more hypoxic regions.

### EXEMPLARY MAGNETOTACTIC SYSTEM WITH RESPECT TO WHICH THE SUBJECT IS PREPARED USING THE PRESENT METHOD

For purposes of illustration, an exemplary magnetotactic system 100 will be described. The method of preparation described herein may be used to prepare a subject for a medical intervention performed using magnetotactic system 100. However, it will be understood that any other magnetotactic system using a magnetic field generated by one or more magnetic field sources (e.g. coils) for navigating magneto-responsive entities once introduced into the body of a subject may benefit from the method of preparation described herein.

The magnetotactic system 100 is for steering magneto-responsive entities in a subject, namely for the purposes of harnessing the magneto-responsive entities for therapy, imagery, diagnostics, etc. For instance, a therapeutic agent, an imagery agent and/or a diagnostic agent may be linked to the magneto-responsive entities, where the entities, once introduced into the subject (e.g. by injection), are steered towards, and may converge at, a targeted zone in the body of the subject, the magneto-responsive entities steered by the magnetic field. As such, the present system enables the steering of these magneto-responsive entities in a subject, once they have been introduced into the subject (e.g., by injection or otherwise). The injection may occur inside the target zone (e.g. intratumoral) or around the target zone (e.g. peritumoral).

The target zone may include one or more hypoxic zones, such as those resulting in a tumor. The target zone may include one or more hypoxic non-vascularized regions in the body of the subject resulting from or associated with, e.g., an ischemic stroke, pulmonary hypertension, ischemic cardiopathy, diabetic retinopathy, etc.

The magnetotaxis system employs a magnetic field mainly for directional control of magneto-responsive entities (without inducing a displacement force to the entities). The fact that the magnetic field from the magnetotaxis system is only intended for directional control or steering and not to provide a propelling or pulling force (although a small if not negligible pulling force may be present close to the magnetic head), translates into the need for a much lower magnitude (intensity) of magnetic field which makes the navigation of magneto-responsive entities technologically possible using much less power.

The system is configured to provide sufficient space (e.g. more than 1.5 meters between the centers of each of the support arms for a human) for the subject laid out on an operating table surrounded by the magnetic sources of the system. The space provided for the subject accommodates subjects of different sizes (such as a child or a fully-grown adult), where the size can also be dictated by the weight of the subject. As different medical interventions require that the subject adopt certain positions (e.g. for pelvic examination: on the subject's back with their legs angled and in the air; for an intervention at the pancreas: on the side with legs spread apart; the supine position, etc.), the space provided to accommodate the subject is also sufficient to set the subject in these different positions such that one or more members of the medical staff (e.g. physicians, nurses, technicians, etc.) can access the subject with little to no hindrance by the structure of the magnetotaxis system.

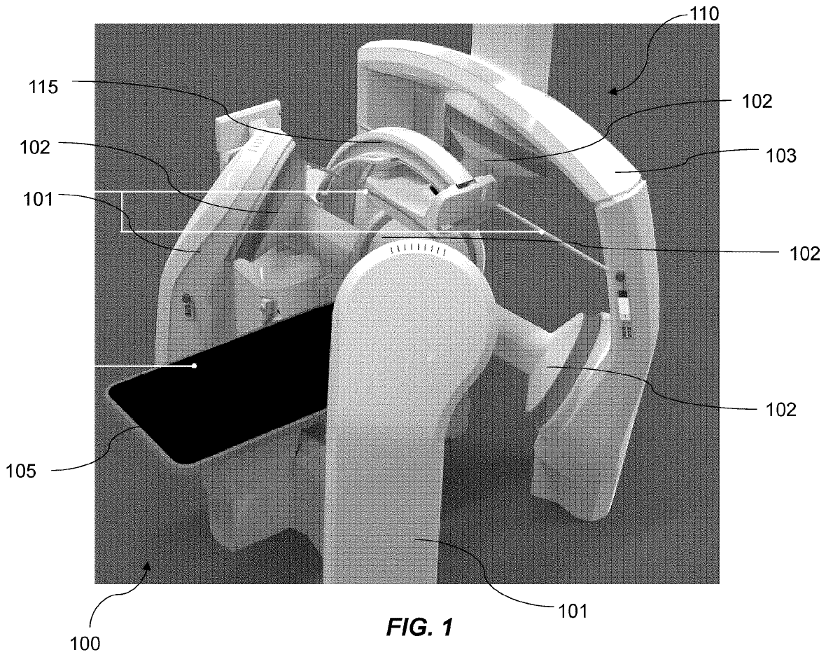

With respect to FIG. 1, the system 100 includes a table 105, six magnetic heads 102 grouped in pairs, and a support structure 110 for supporting the magnetic heads 102 in a given configuration.

The system 100 may include an imaging device 115 such as an x-ray image intensifier such as a C-arm.

The magnetic heads 102 generate the magnetic field for steering the magneto-responsive entities. As such, the magnetic heads 102 each have one or more magnetic coils surrounding a ferromagnetic core that generates a magnetic field when a current is passed therethrough. The magnetic heads 102 are paired together, thereby forming at least three pairs. Each of the pairs of magnetic heads 102 is defined by one axis of three axes that are orthogonal with one another. In embodiments, none of the three axes that define the orientation of the magnetic heads 102 is parallel with the floor, and none of the three axes is parallel with any of reference axes x, y and z (where the x-axis is parallel with the floor). The pairs of magnetic heads 102 may establish a point in a body of the subject where the magneto-responsive entities will navigate towards and converge, using, for instance, the techniques described in U.S. Pat. No. 9,905,347. However, due to the size of the magnetic heads 102, the requirement of providing sufficient space to fit a subject amongst the magnetic heads 102 such that the convergence point or magnetic field generated by the magnetic heads 102 can be generated in the body of the subject, and to provide sufficient space to allow a physician to access the subject when lying on the table 105 (considering that the space occupied by the subject and physician may depend on the nature of the procedure to be performed on the subject, as explained herein), a support structure may be needed.

Support structure 110 supports each of the magnetic heads 102 in their given configuration where the magnetic heads of each pair of magnetic heads 102 are positioned in opposition to one another along an axis oriented orthogonally with respect to the axes of the other pairs of magnetic heads 102 for generating a magnetic field capable of steering the magneto-responsive entities in three dimensions. The magnetic heads 102 of each pair of magnetic heads 102 are facing one another. The support structure 110 may include a support arch 103 and two support arms 101.

The support arch 103 and the two support arms 101 may be positioned at or near opposite ends of the table 105 such that the magnetic heads 102 supported thereby can be fixed in a proper orientation for generating the necessary magnetic field adapted for creating a torque along each of the three axes x, y and z.

Each of the support arms 101 may support one magnetic head 102 of a different pair of magnetic heads 102. The magnetic head 102 may be positioned at or near a top of the support arm 101. The support arm 101 may be shaped such that the support arm 101 is arched downward, or may have a vertical portion and a portion for receiving a magnetic head 102 that is at an angle with the vertical portion of the support arm 101, thereby orienting the magnetic head 102 at least slightly downward, towards the table 105. As such, the shape of the support 101 may be determined to position the magnetic head 102 attached thereto in a proper angle to face the corresponding other magnetic head 102 of the pair of magnetic heads 102. The orientation of the magnetic head 102 may be such that the magnetic head 102 supported by the support arm 101 is facing the corresponding magnetic head 102 of the magnetic head pair, as described herein. In some embodiments, each of the support arms 101 may be connected to one another at a base portion (e.g. joins the base of the table 105.) In other embodiments, each of the support arms 101 may be separate from the table 105.

In some examples, the support arms 101 may be configured to move away and towards the table 105, e.g. in order to create additional space for placing the subject on the table 105. For instance, the support arms 101 may be positioned on rails, where the support arms 101 may slide along the rails to create additional space.

In some examples, the magnetic heads 102 may be pivotably attached to the support arms 101 or the support arch 103, such that they may be displaced in order to further create space between the magnetic heads 102, e.g. for placing the subject on the table 105.

The support arch 103 may support one or more magnetic heads 102. As is shown in the example of FIG. 1, the support arch 103 supports three magnetic heads 102. A first magnetic head 102 supported by the support arch 103 is paired with a magnetic head 102 supported by the first support arm 101, a second magnetic head 102 supported by the support arch 103 is paired with a magnetic head 102 supported by a second support arm 102, and a third magnetic head 102 supported by the supper arch 103, centered on the support arch 103, is paired with the magnetic head 102 positioned under the table 105.

The support arch 103 has two leg portions, contacting the ground and that are substantially vertical (the leg portion may have a slight curve or bend as illustrated, for instance, in FIG. 1) and an arch portion that is curved, interconnecting the two leg portions of the support arch 103.

Each of the leg portions of the support arch 103 may receive one magnetic head 102. The magnetic head 102 may be located near or at the bottom of the leg portion of the support arch 103. The width or thickness of the leg portion of the support arch 103 may be greater towards the bottom in order to support the magnetic head 102 (e.g. may not be uniform). In some examples, the leg portion of the support arch 103 may include a user input interface for controlling the system (e.g. the generating of a magnetic field using the pairs of magnetic heads 102).

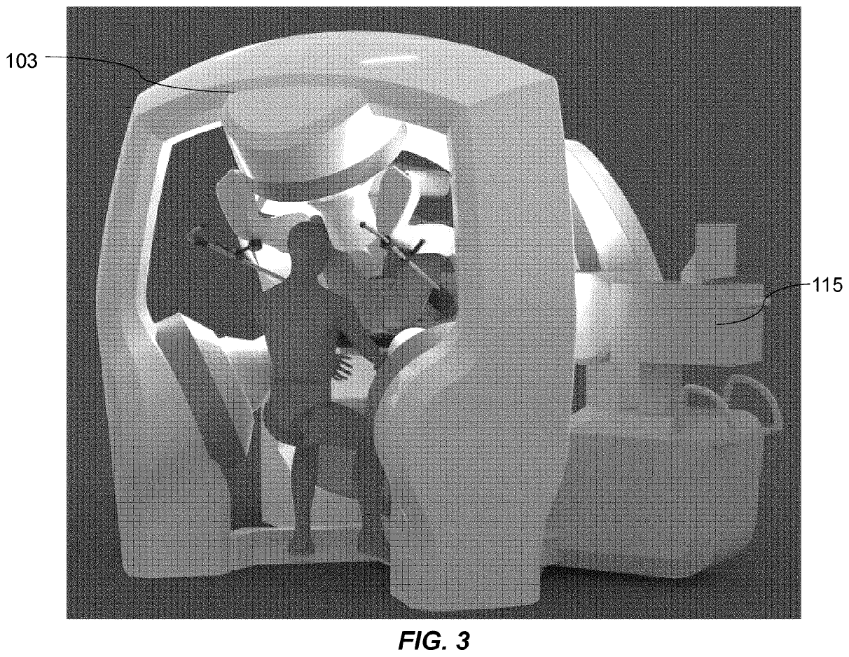

The arch portion of the support arch 103 creates a space as a result of the elevation of the middle of the arch for an intervening physician to access the subject, as shown in FIG. 3.

In some examples, instead of a pair of support arms 101, the system 100 may include a second support arch for receiving the two magnetic heads 102 instead of the support arms 101 (not shown).

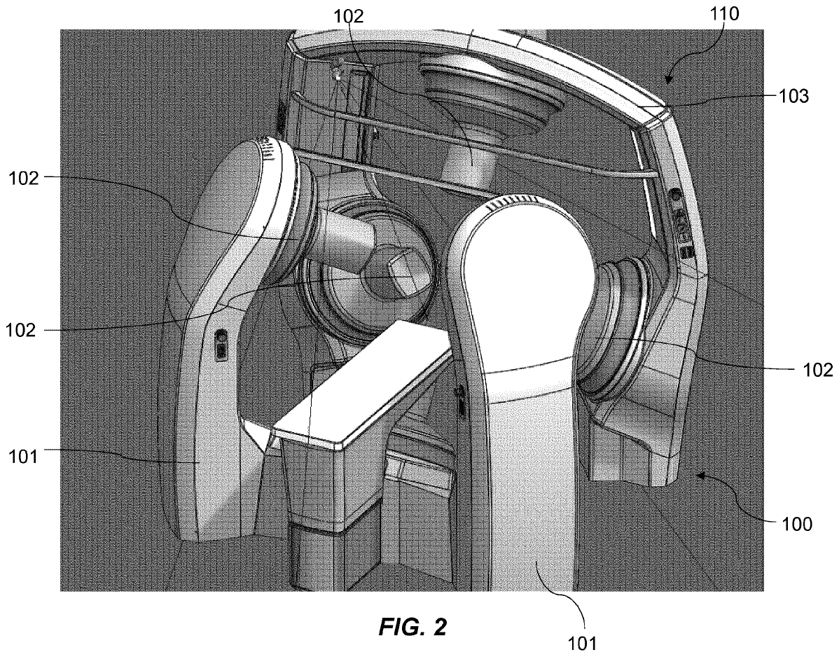

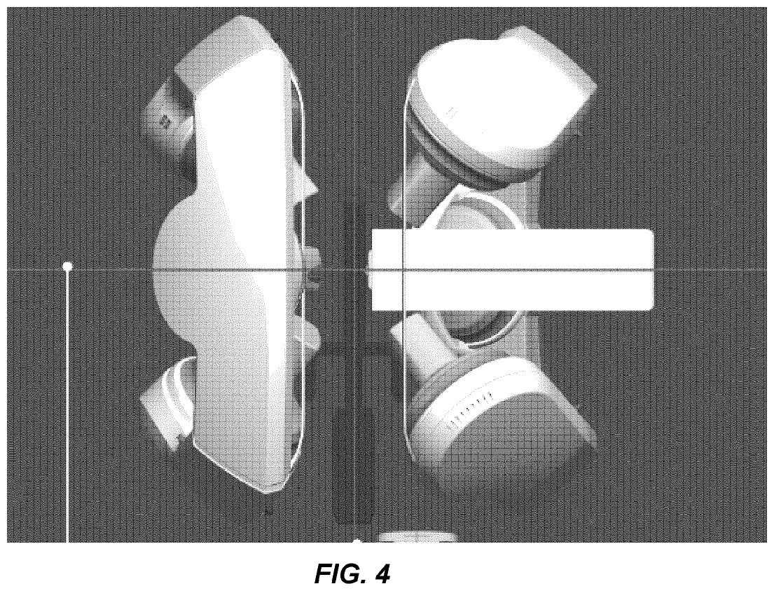

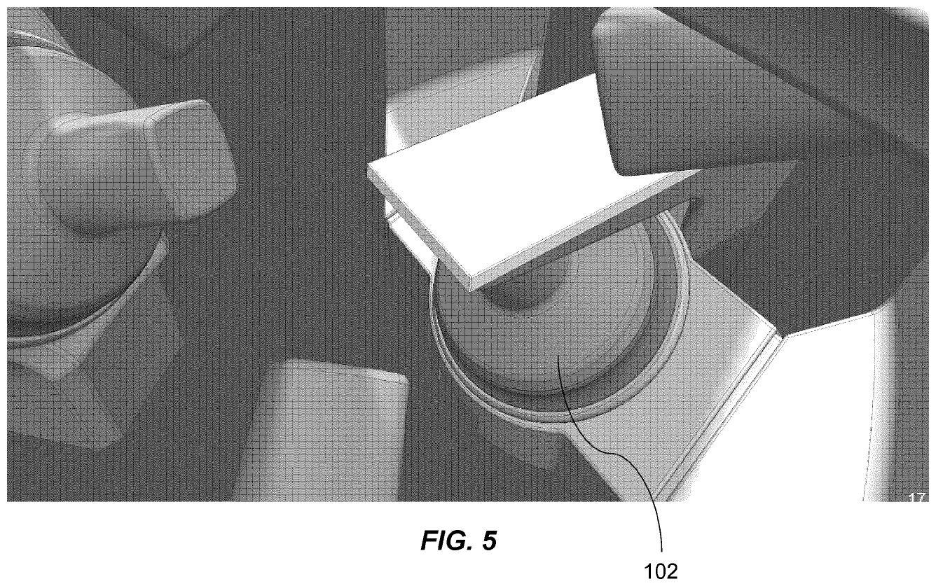

It will be understood that the orientation and position of the magnetic heads 102 shown in FIGS. 1-5 illustrate one example of same that was developed for meeting the size constraints and to generate sufficient space for the subject and intervening members of medical staff to perform certain medical interventions, as described herein, where certain medical interventions require that the subject be laid in a given position.

The table 105 is adapted to receive the subject into whom will be introduced the magneto-responsive entities. As such, the length of the table 105 may be sufficient to receive one of possible subjects of different heights and widths lying down (e.g. over 6.5 feet). The table 105 may be adapted to move back-and-forth, side-to-side and/or up-and-down (i.e. along one or more of the three axes x, y, and z). In some embodiments, the table 105 may also be configured to rotate clockwise and/or counter-clockwise. The movement of the table 105 may be controlled by a user using a user input interface, such as the one appearing on the leg portion of the support arch 103 in FIG. 1. In other embodiments, the system 100 may be controlled remotely (e.g. using a remote control that communicates wirelessly with the system 100; a computing device such as a tablet computer or smartphone including program code for controlling the system 100 when executed by the processor of the computing device, etc.)

The table 105 may be layered with a thin mattress or cushion to provide comfort for the subject. In some examples, the table 105 may have extensions for receiving the arms of the subject.

The imaging device 115 is configured to obtain information on the anatomy of the subject (imaging of the inside of the subject). In some embodiments, this information may be used to monitor the progress of the magneto-responsive entities in the subject, determine the location of the target zone towards which the magneto-responsive entities will be steered, to provide visuals for the purpose of a medical intervention such as a surgery, etc. The imaging device 115 may be an X-ray image intensifier, such as a C-arm. However, it will be understood that other imaging devices may be used without departing from the present teachings.

In some examples, as shown in FIG. 1, the imaging device 115 may fit in a space between the support arm 101 and the support arch 103. When the imaging device 115 is a C-arm, the arch of the C-arm may arch around the table 105 such that the C-arm can gather information on the subject lying on the table 105, where, e.g., the table 105 may move up and down with respect to the C-arm such that the C-arm can provide imaging data along a length of a body of the subject. In some embodiments, the imaging device 115 may be displaced manually or remotely, having, for instance, a pair of wheels for rolling the imaging device 115 into different positions.

Reference is now made to FIG. 3 to further illustrate an exemplary system for steering magneto-responsive entities as described herein. The layout of the system 100 may be dictated by the size of the magnetic heads 102, the positioning on the surface to receive a subject such that the pairs of magnetic heads 102 may generate a magnetic field for steering magneto-responsive entities introduced into the subject, and finally to allow a physician to have access to the subject in order to conduct a medical intervention.

In some embodiments, the dimensions of the support arch 103, the orientation of the leg portions of the support arch 103 and the orientation of the magnetic heads 102 fixed thereon are to allow access to a physician to fit within the space defined by the arch 103. Moreover, for certain medical procedures, the position of the subject may result in the subject occupying more space. For instance, for a medical intervention pertaining to rectal cancer, the subject's legs are suspended and spread open as illustrated in FIG. 3. As such, the layout of the system 100 takes into account the space occupied by the subject and the physician during different medical procedures, and more particularly with respect to those where the subject occupies a greater amount of space due to the position of the subject. The magnetic head 102 positioned at the top of the support arch 103 faces towards the table 105, and mirroring the orientation of the magnetic head 102 positioned under the table 102. By having the magnetic head 102 positioned at the center of the support arch 103 be oriented towards the table 105, thereby not being entirely vertical, this provides sufficient space far a seated physician to be positioned under the support arch 103, the angled magnetic head 102 providing more space under the support arch 103.

The leg portions of the support arch 103 are not parallel, e.g. may define an angle of 90 degrees or less with respect to one another.

In some examples, for treating breast cancer, the system 100 is adapted to generate a target zone near or at the breast while the subject is lying on the table 105. This may be further achieved by having the table 105 move up and/or down in order to reposition the subject with respect to the magnetic heads 102.

Depending on the nature of the intervention, the subject and the intervening members of the medical staff take up varying amounts of space. The layout of the system is configured to provide for the space occupied by the members of the medical staff. This is because the system 100 can be used to prepare for, and/or for use with, many possible medical interventions.

In an exemplary embodiment, the distance between the first and second support arms 101, measured from their respective centers at their base, may be of 1698 mm. The table 105 may be position at the midpoint between the first and second support arms 101. An exemplary support arch 103 may have a height of 2540 mm, a width of 1593 mm measured from the respective centers of the base of the leg portions of the support arch 103. It will be understood that other possible dimensions may be possible without departing from the present teachings.

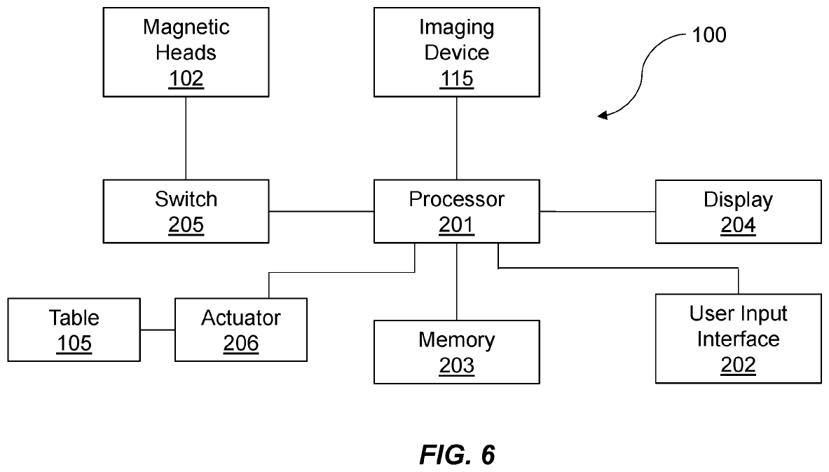

Reference is now made to FIG. 6, illustrating an exemplary computer architecture of an exemplary system for controlling magneto-responsive entities 100, interacting with certain of the components of the system 100.

The system 100 includes a processor 201, memory 203 (e.g. on a printed circuit board—PCB) and a controller 205 (e.g. including a current amplifier).

The system 100 may include a user input interface 202 and/or a display 204. The computer may include an actuator 206.

The processor 201 may be a programmable processor. In this example, the processor 201 is shown as being unitary, but the processor may also be multicore, or distributed (e.g. a multi-processor).

The computer readable memory 203 stores program instructions and data used by the processor 201. The memory 203 may include non-transitory storage to store the program instructions. The computer readable memory 203, though shown as unitary for simplicity in the present example, may comprise multiple memory modules and/or caching. In particular, it may comprise several layers of memory such as a hard drive, external drive (e.g. SD card storage) or the like and a random access memory (RAM) module. The RAM module may store data and/or program code currently being, recently being, or soon to be processed by the processor 201 as well as cache data and/or program code retrieved from non-transitory memory, e.g., a hard drive. A hard drive may store program code and be accessed to retrieve such code for execution by the processor 201 and may be accessed by the processor 201 to store magnetic head 102 sequences for generating a convergence point for the magneto-responsive entities, and imaging data from the imaging device 115, as explained herein. The memory 203 may have a recycling architecture for storing, for instance, imaging data from the imaging device 115, coordinates for steering the magneto-responsive entities, etc., where older data files are deleted when the memory 203 is full or near being full, or after the older data files have been stored in memory 203 for a certain time.

The user input interface 202 is in communication with the processor 201. The user input interface 202 allows for a user to provide input to the system 100, e.g., for controlling the magnetic heads 102, moving the table 105, activating the imaging device 115. The user input interface 202 may be one or more of a touchscreen, one or more knobs, a keyboard, a mouse, a joystick, etc. As shown in FIG. 1, the user input interface 202 may be integrated to a leg portion of the support arch 103 (however, the user input interface 202 may be separate from the support arch 103 or support structure 110, or may be integrated, e.g., to a support arm 101).

The processor 201, the memory 203 and the user input interface 202 may be linked via BUS connections.

The display 204 provides visual information to the user of the system 100, such as imaging data generated by the imaging device 115, values for the strength of the magnetic field generated by one or more pairs of magnetic heads 102, the location of the convergence point for steering the magneto-responsive entities, overlaid, e.g., over an image of the subject generated by, for instance, the imaging device 115. The display 204 may also provide a graphical user interface for the user for controlling the system 100. The display 204 may also include a functionality of a user input interface 202, being configured as a touchscreen.

The one or more actuators 206 controls the position of the table 105. The one or more actuators 206, upon receiving commands from the processor 201, may cause the table to move up-or-down, side-to-side, forwards-or-backwards, or rotate. The one or more actuators 206 may be any combination of pneumatic, hydraulic, supercoiled, electric, rotary, linear, etc.

One or more controllers 205 (e.g. including current amplifiers) may be present for controlling the flow of current to the one or more magnetic heads 102, in order to cause the generation, or modifying the generation of the magnetic field generated by the one or more magnetic heads 102 when powered, as described in further detail below.

The user may control the system 100 by providing input via the user input interface 202. For instance, the user may provide input for activating one or more of the magnetic head pairs (or this may be done implicitly by the user designating a target in the subject). The processor 201 receives the input for turning on one or more of the magnetic heads, and sends commands to cause the controller 205 to open one or more switches and/or modulate current being directed to the one or more magnetic head pairs (e.g. through current amplifiers), as described in further detail below. In some embodiments, where the user provides a target for steering the magneto-responsive entities, the processor 201 may retrieve from memory 203 one or more commands or functions to identify the appropriate magnetic head pairs 102 to turn on, the appropriate current to be provided to the one or more magnetic heads, and/or if the current is to remain constant, and/or if the magnetic field generated by each of the magnetic heads 102 is to remain constant or is to fluctuate in a time multiplexed manner, as explained in U.S. Pat. No. 9,905,347. In some embodiments, upon the user selecting a target in a subject for the steering of the magneto-responsive entities, the processor 201 may also generate one or more commands transmitted to the actuator 206 in order to displace the table 105, and the user positioned thereon, for positioning the subject with respect to the convergence point that may be generated by the pairs of magnetic heads 102.

The user may also provide input via the user input interface 202 to displace the table 105. This input is received by the processor 201, where the processor 201 retrieves from memory 203 commands or functions to calculate table adjustment(s) and sends commands corresponding to the user input to the actuator 206 to cause the table 105 to be displaced.

The user input interface 202, processor 201 and memory 203 may be used to control the imaging device 115. However, in some embodiments, the imaging device 115 may have a separate computer, including a user input interface, for interacting with same.

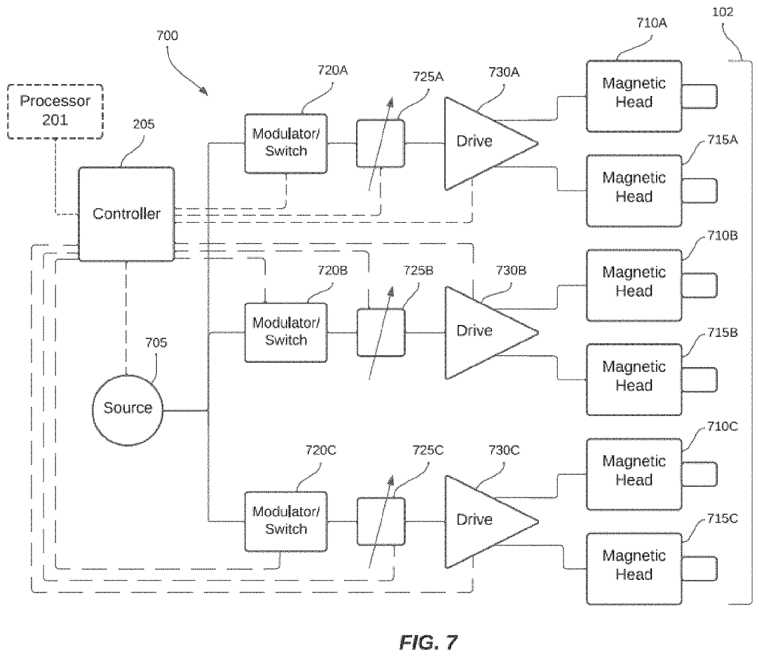

FIG. 7 depicts a controller 205 in communication with the components of a magnetic head subsystem 700. As explained above, the controller 205 is in communication with the processor 201 (see FIG. 6). The processor 201 sends commands to the controller 205 and may receive various types of data from the controller 205 relating to the state of the components of the magnetic head subsystem. The controller 205 controls the components of the magnetic head subsystem 700 to provide drive signals to the magnetic heads 102.

The magnetic heads 102 may be configured as three pairs of opposing heads, each pair aligned along one of three orthogonal axes. For example, a first pair of opposing magnetic heads (710A, 710B) may be positioned along a first axis, a second pair of opposing magnetic heads (715A, 715B) may be aligned along a second axis, and a third pair of opposing magnetic heads (720A, 720B) may be aligned along a third axis.

The magnetic head subsystem 700 includes a source 705, e.g., a radio frequency oscillator, to provide current to the magnetic heads 102. The output of the source 705 may be fed to a number of drive component chains, each of which may include a modulator/switch (720A, 720B, 720C), a phase shifter (725A, 725B, 725C), and a drive (730A, 730B, 730C). In disclosed embodiments, there may be a separate source 705 for one or more of the drive component chains, e.g., a separate source for each drive component chain.

In the example depicted, each pair of magnetic heads (e.g., 710A, 715A) has an associated chain of drive components (e.g., 720A, 725A, 730A). In disclosed embodiments, a full chain of drive components may be provided for only a subset of the pairs of magnetic heads. For example, if only two of the three pairs of magnetic heads are to receive modulated signals, then the drive (e.g., 730C) of the third pair of magnetic heads (e.g., 710C, 715C) could be in communication with the source 705 without an intervening modulator/switch (e.g., 720C). Similarly, in disclosed embodiments, the phase shifter (e.g., 725C) may be omitted from a respective drive component chain if the corresponding pair of magnetic heads (e.g., 710C, 715C) does not require a phase-shifted signal at the input of the drive (e.g., 730C).

The modulator/switch (720A, 720B, 720C) receives the output of the source 705 and performs modulation or switching to generate a desired waveform, e.g., a square-wave or pulsed waveform. One of ordinary skill in the art would understand that a modulator can be implemented with various circuit architectures, including in a form that is, in essence, a switch. Hence, the terms “modulator/switch,” “modulator,” and “switch” are used interchangeably in the present specification, including the claims. The output of the modulator/switch (720A, 720B, 720C) is received by a phase shifter (725A, 725B, 725C), which applies a determined phase shift to the drive signal, e.g., by applying a delay to the signal. In this example, the phase shifter (725A, 725B, 725C) is depicted as a separate component. However, one of ordinary skill in the art would understand that a phase shift could instead be provided by another component, such as the modulator/switch.

Each drive component chain includes a drive (e.g., 730A) which outputs a drive current to a pair of the magnetic heads (e.g., 710A, 715A). The drive (e.g., 730A) receives a drive signal from the source 705—possibly after passing through a modulator/switch (e.g., 720A) and/or a phase shifter (e.g., 725A). The drive (730A, 730B, 730C) may be, for example, an amplifier which amplifies a received drive signal to generate a high-power drive current (or voltage) to drive a pair of the magnetic heads. In disclosed embodiments, the drive (e.g., 730A) may output drive currents of opposite polarity to respective ones of the pair of magnetic heads (e.g., 710A, 715A). One of ordinary skill in the art would understand that a separate drive may be used for each magnetic head.

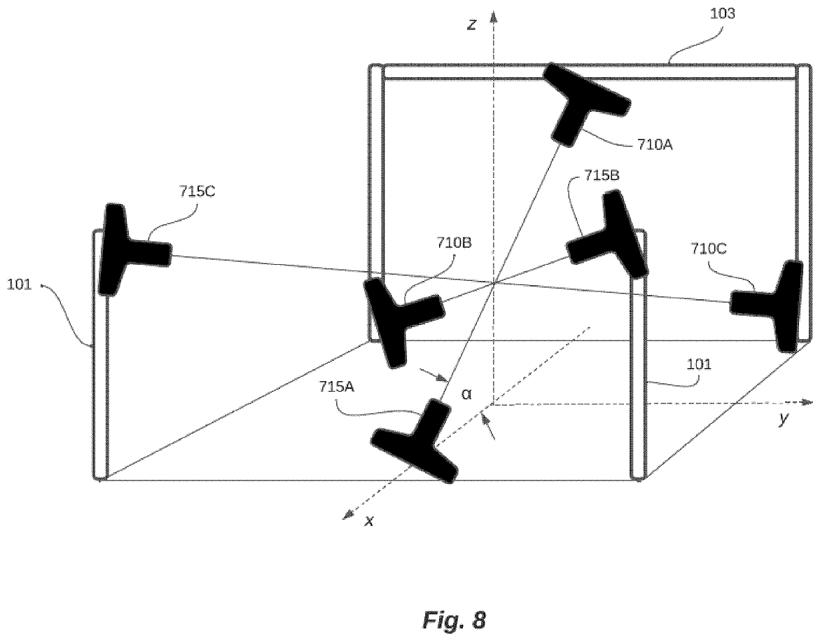

FIG. 8 depicts a geometric relationship of positions and orientations of the magnetic heads. As discussed above, the system 100 may include three pairs of magnetic heads (710A-C, 715A-C). The magnetic heads 102 of each pair of magnetic heads may be positioned opposed to each other (i.e., facing each other) along a particular one of three substantially orthogonal axes. By the term “substantially orthogonal” it is meant that the axes need not be precisely orthogonal—the system 100 can be calibrated for axes which vary somewhat (e.g., +/−5% or +/−10%) from orthogonality.

The magnetic heads (710A-C, 715A-C) are held in position by the support arms 101 or the support arch 103 or positioned on the floor, i.e., under the table 105 (see FIG. 1). In the example depicted, a first pair of magnetic heads (710A, 715A) is positioned such that a first magnetic head 710A is affixed to the support arch 103, while the second, opposing magnetic head 715A is affixed to the floor or to a base in contact with the floor. A second pair of magnetic heads (710B, 715B) is positioned such that a first magnetic head 710B is affixed to the lower portion of one of the legs of the support arch 103, while the second, opposing magnetic head 715B is affixed to one of the support arms 101. Similarly, a third pair of magnetic heads (710C, 715C) is positioned such that a first magnetic head 710C is affixed to the lower portion of the other leg of the support arch 103, while the second, opposing magnetic head 715C is affixed to the other support arm 101.

As a reference, a set of orthogonal axes may be defined relative to the plane of the floor, with the x-axis running along the floor in a direction away from the support arch 103 and toward a mid-point between the support arms 101, i.e., from the head to the foot of the table 105 (see FIG. 1), the y-axis running along the floor in a direction perpendicular to the x-axis, and the z-axis running in a vertical direction toward the ceiling. In the example depicted, none of the first, second, or third axes of the pairs of magnetic heads (710A-C, 715A-C) is orthogonal to the x, y, or z axes.

Specifically, the second magnetic head 715A of the first pair of magnetic heads (710A, 715B) is positioned to form an angle, a, with respect to the x-axis (i.e., with respect to the plane of the floor), where a is less than 90°. In some examples, α may be 54 degrees. As such, the axis of the first pair of magnetic heads (710A, 715A) forms an angle of 90°—α with the z-axis (i.e., the vertical axis). The axis of the second pair of magnetic heads (710B, 715B) extends from one of the support arms 101 to the lower portion of a leg of the support arch 103 and, thus, is not parallel to the x or y axes (or the z-axis) and, consequently, is not parallel or perpendicular to the table 105 (see FIG. 1). In some embodiments, other angles may be around 25 degrees (e.g. 23.9 degrees). Similarly, the axis of the third pair of magnetic heads (710C, 715C) extends from the other support arm 101 to the lower portion of the other leg of the support arch 103 and, thus, is not parallel to the x or y axes (or the z-axis) and, consequently, is not parallel or perpendicular to the table 105 (see FIG. 1). In some embodiments, some of the angles between an axis of a pair of magnetic heads and x. y or z may be equal (e.g. around 25 degrees, such as 23.9 degrees).

By virtue of the geometric relationship described above, it can be seen that the first magnetic head 710A of the first pair of magnetic heads (710A, 715A) is not directly over the center of the table 105 (see FIG. 1) where it would interfere with positioning of a subject on the table 105. Furthermore, the arrangement of the second pair of magnetic heads (710B, 715B) and third pair of magnetic heads (710C, 715C) is such that they are not positioned at the head or the foot of the table 105 (see FIG. 1) where they would interfere with access to the subject by a physician. Moreover, the second pair of magnetic heads (710B, 715B) and third pair of magnetic heads (710C, 715C) are displaced vertically (i.e., above or below) relative to the level of the table 105 (see FIG. 1). The geometric relationship also allows for three of the six magnetic heads to be mounted on the support arch 103 and two of the magnetic heads to be mounted on two respective support arms 101, which reduces the complexity of the support structure.

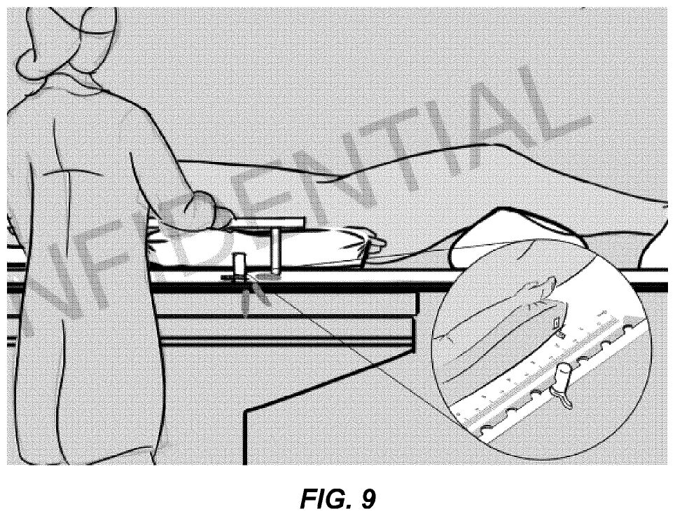

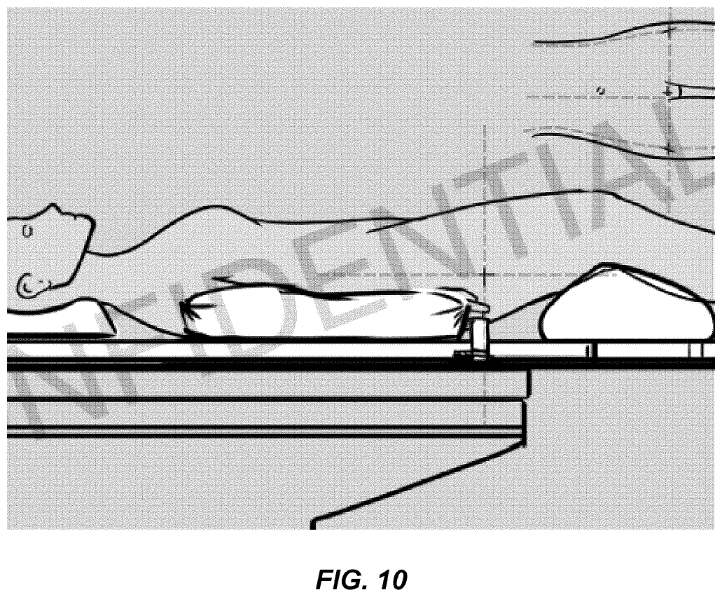

In some embodiments, as shown in FIGS. 9 and 10, the floor, ceiling and/or walls of the room in which the system 100 is located may be used to support one or more of the magnetic heads, where the magnetic heads are positioned with respect to one another in a similar manner as described in FIG. 8. As such, as shown in FIG. 9, as one of the magnetic heads is joined to the ceiling, and two of the magnetic heads are joined to the floor, the exemplary system 100 of FIG. 9 does not include the arch for providing support to these three magnetic heads, as the ceiling and floor replace the arch for providing support. FIG. 10 shows the three magnetic heads of FIG. 9 that were joined to an arch in FIG. 1, where two of the three magnetic heads are joined to the floor, and one of the three magnetic heads is joined to the ceiling. Necessary wiring for the system, including the magnetic heads, may respectively be passed on or under the floor, and on or in the ceiling. Similarly, in some examples, walls may also be used to support the magnetic heads that were joined to the support arms in FIG. 1, thereby replacing the support arms as mechanisms for supporting the magnetic heads (not shown).

### EXEMPLARY METHOD OF PREPARING A SUBJECT FOR A MEDICAL INTERVENTION PERFORMED USING A MAGNETOTACTIC SYSTEM

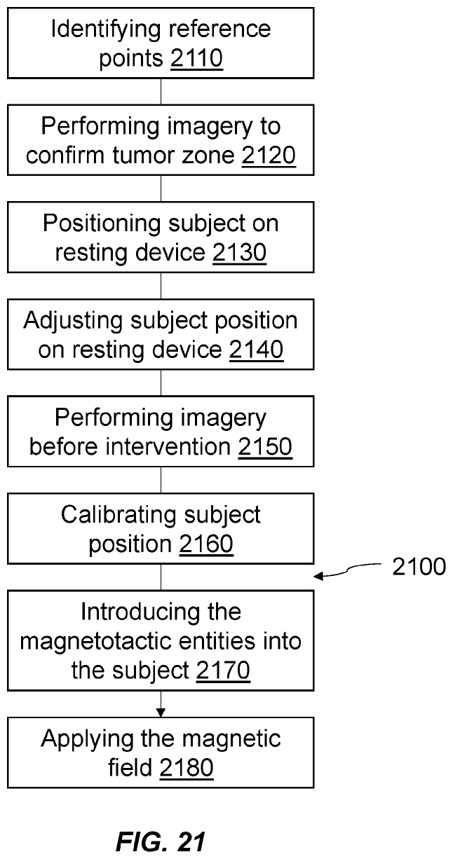

Reference is now made to FIG. 21, illustrating an exemplary method 2100 of preparing a subject for a medical intervention using a magnetotactic system (e.g. such as magnetotactic system 100). For purposes of illustration, reference will be made to the magnetotactic system 100. However, it will be understood that other magnetotactic systems in accordance with the present teachings may be used.

In method 2100, reference is made to a tumor as being the target for the medical intervention. However, it will be understood that other locations or organs in the body of the subject may be the target of the present medical intervention, to which the magneto-responsive entities are to be directed, such as the heart for ischemic heart disease, the lungs for pulmonary hypertension, the brain for acute ischemic stroke, the eyes for diabetic retinopathy, etc.

Prior to the medical intervention (e.g. one or more days prior to the medical intervention), the subject may be positioned on a resting device (e.g. a table with an immobilization board, such as the ALTA™ board from Q-FIX™, as shown in FIG. 9) and immobilized with immobilizing equipment. The position of the immobilizing equipment may be identified or docketed to permit laying out of the equipment on the resting device in a same configuration and position for enabling the subject to be reinstalled on same with consistency. Exemplary immobilizing equipment may be, for instance, vacuum cushions, couch top pads, etc. (such as the VACQFIX™ Vacuum Cushions).

Following immobilization, initial reference points are identified on the subject, to generate a reference system on the body of the subject for the purpose of imagery. These initial reference points are placed on the subject with respect to the isocenter of the device that will perform imagery of the subject to ascertain the location of the target zone in the subject (e.g. MRI machine, by performing a CT scan, a PET scan) where the center of the coordinate system of the initial reference points is to align with the isocenter of the imagery device.

An example of adding the initial, imagery reference points is illustrated in FIG. 10.

As such, in some examples, the reference points may form a coordinate system, where some of the reference points may be positioned on the sides of the subject, as shown in FIG. 10, in each of the three axes, x, y and z. The reference points define the center of the coordinate system (at 0,0,0) corresponding to the isocenter of the imagery machine. In some examples, reference points may be positioned on a front, a back, a first side and/or a second side (left or right side) of the subject. In one example, a minimum of three reference points are to be defined on the subject in order to define the coordinate system.

The addition of the reference points may be performed one or more days prior to the medical intervention.

In some implementations, the reference points may be fiducial markers (tattoos) added to (e.g. drawn on) the body of the subject. The reference points may also be anatomical features of or on the body of the subject (e.g. belly button, nipples, a mole, a bone, a mass indicative of a location of the target zone—a tumor, etc.) In some examples, the reference points may include lines (e.g. stippled lines) added to the skin of the subject. In some embodiments, the reference points may be, or may include, certain compounds, such as metals, for enabling the visualization of the reference points when performing medical imagery of the subject. In some embodiments, these compounds for the purposes of visualization during imagery may be added at a later time, as described herein.

The position of the subject with respect to the immobilizing equipment may also be determined, where markings can be added to the immobilizing equipment indicative of a location of a body part of the subject (e.g. head of a subject, shoulders of the subject, pelvis of the subject, etc.) when resting on the immobilizing equipment. In some embodiments, further reference points may also be added to the subject indicative of the location of some of the immobilizing equipment with respect to the subject. These further reference points may enable the repositioning of the subject onto the immobilizing equipment, for the medical intervention, where the further reference points may guide the positioning of the subject onto the immobilizing equipment.

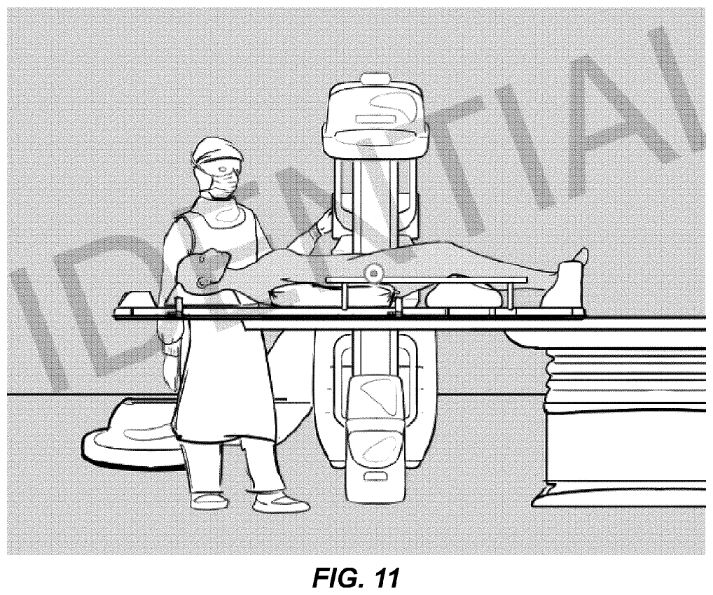

Imagery is performed to identify the position of the tumor in the subject at step 2120 (in some cases, on a subsequent day to the day on which step 2110 was performed). The initial reference points may be used to position the subject for the purposes of imagery, where the reference system found on the subject may align the patient with respect to the isocenter of the machine device. Imagery may be obtained by performing an MRI scan, a PET scan, a CT scan, etc., of the subject. Imagery markers may be added to the subject at the initial reference points to identify the initial reference points in the scans obtained during imagery, enabling the visualization of the initial reference points during imagery, e.g., with respect to the tumor. The imagery markers may be, for instance, pieces of metal attached to the subject, etc., as shown in FIG. 11.

In some embodiments, the initial references points are defined on the subject using a laser system of the imagery device to align a location of the subject for which imagery is to be performed, with the isocenter of the imagery device. For instance, in some instance, when the tumor is suspected to be present in the pancreas, the laser system may be positioned to grossly target a pancreas region of the subject. The laser system of the imagery device may include a set of lasers that produce beams of light that follow different axes, used to plot out a coordinate system on the subject where the light, produced by the lasers, appears on the subject. A location where the beams of light cross may define the center of the coordinate system, to align with the isocenter of the imagery device.

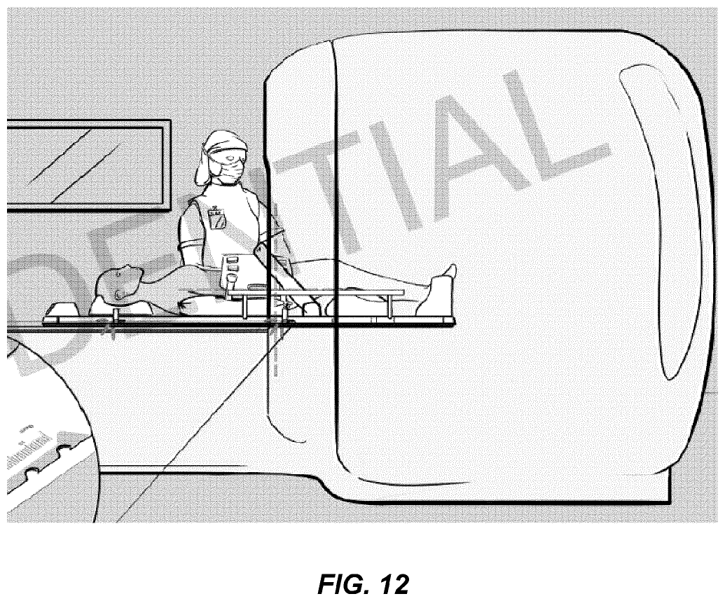

With the imagery markers added to the subject, imagery is performed, as shown in FIG. 12 (illustrating an exemplary magnetic resonance imaging (MRI) machine for the purpose of imagery).

The imagery enables the visualization of the tumor in the patient. A position of the tumor can be defined in three dimensions (in three axes). However, it will be understood that the position of the tumor may also be defined in two dimensions (e.g. when the tumor is on a surface of the subject).

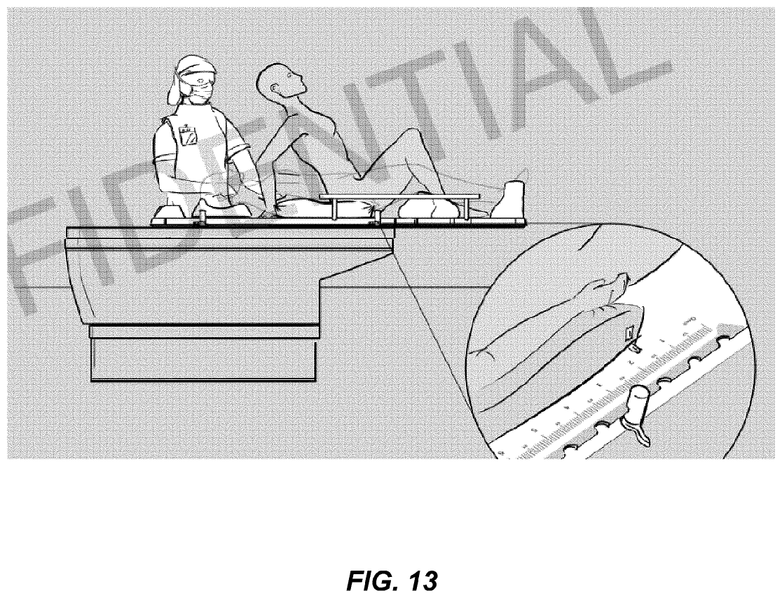

Prior to the medical intervention, on the day thereof, the subject is repositioned on the immobilizing equipment located on a resting device at step 2130 (e.g. a table), as shown in the example of FIG. 13. In some examples, the markers added to the immobilizing equipment and/or the corrected reference points added to the subject may be used to position the subject in a configuration with respect to the immobilizing equipment that was determined at step 2110.

The coordinate system defined by initial reference points is compared to the position of the tumor, as identified during imagery, and a difference between the initial coordinate system and the position of the tumor is measured. Differences in one or more of the x, y and z axes are accounted for between the position of the tumor and the location of the center of the coordinate system of the initial reference points, the isocenter, (e.g. 0,0,0) defined by the initial reference points. This difference can be defined as a vector in three axes (x, y, z,) and defines an isocenter shift (a translation performed between the center of the coordinate system defined by the initial reference points, and the center of the area of the tumor identified through imagery).

Treatment reference points may be added to the subject, applied as a function of the imagery information, mapping out the tumor identified through the imagery performed at step 2120. The treatment reference points may be added to the subject prior to the medical intervention using the magnetotactic system, by starting at the isocenter defined by the initial references points identified on the subject, and performing a translation corresponding to the isoshift (e.g. in x, y, z). For purposes of accuracy, the translation may be performed using a laser system, where a set of lasers may be used to visualize the location of the tumor in the subject, the light from the lasers appearing on the exterior (e.g. skin) of the subject. The lasers may be oriented around the subject such that the light from the lasers cross, the laser light aligned with different axes. The treatment reference points (or additional reference points) may then be added based on where the light of the lasers appears on the subject, the laser light guiding the addition of the additional reference points (e.g. fiducial markers).

In some implementations, the treatment reference points may be fiducial markers (tattoos) added to (e.g. drawn on) the body of the subject. The treatment reference points may also be anatomical features of or on the body of the subject (e.g. belly button, nipples, a bone, a mass indicative of a location of the target zone—a tumor, etc.) In some examples, the treatment reference points may include lines (e.g. stippled lines) added to the skin of the subject. In some embodiments, the treatment reference points may be, or may include, certain compounds, such as metals, for enabling the visualization of the reference points when performing medical imagery of the subject. In some embodiments, these compounds for the purposes of visualization during imagery may be added at a later time, as described herein.

A difference in position between the initial reference points and the treatment references points, identifying a corrected coordinate system, corresponds to the calculated isocenter shift. As such, the corrected coordinate system defined by the corrected reference points relates to the tumor detected during imagery.

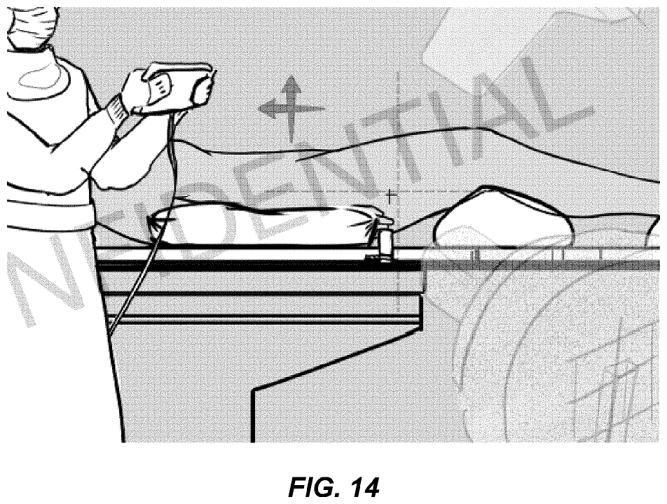

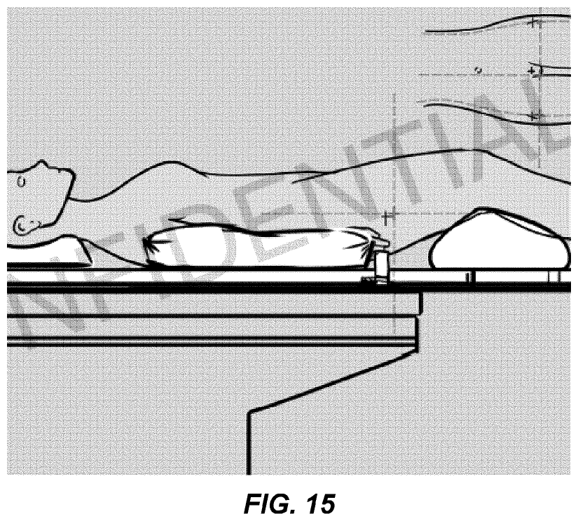

As illustrated in FIG. 14, the medical practitioner may adjust the position of the resting device, and/or the subject on the resting device, to compensate for the difference in position between the original reference points applied to the subject at step 2140, and the treatment reference points added to the subject following imagery of the tumor, the treatment reference points indicative of the location of the tumor (following the isocenter shift). This isocenter shift results in the tumor, defining the target zone, being located at a position appropriate to be aligned with the isocenter or the 3D convergence point of the magnetic heads of the magnetotactic system, or at least within a target zone of the magnetactic system in which guidance of the magneto-responsive entities to the target zone is possible using the magnetic field generated by the magnetic coils of the magnetotactic system. FIG. 15 illustrates the difference in position between the isocenter of the coordinate system (0,0,0) defined by the original reference points, added at step 2110, and the treatment reference points, added at step 2120, defining a corrected coordinate system with its center corresponding to the position of the tumor (x, y, z), the difference corresponding to the isocenter shift.

The positioning of the subject in the magnetotactic system may be performed using a laser system. The lasers are used to guide the positioning of the subject to align the center of the treatment reference points with the isocenter of the magnetotactic system. For instance, the laser system may include a set of lasers that produce beams of light that follow different axes, used to plot out a coordinate system on the subject where the light, produced by the lasers, appears on the subject. A location where the beams of light cross may define the isocenter of the magnetotactic system.

In some examples, a position of reference points with respect to the tumor may then be confirmed (i.e. by determining if the reference points have moved with respect to the tumor from the first time the reference points were added to the subject—this could be as a result of bloating of the subject, emptying of the intestine, etc.). The confirmation of the reference points with respect to the tumor may be performed any number of times prior to the medical intervention.

If the subject is not already positioned in the magnetotactic system, the subject on the resting device is moved to the magnetotactic system.

The subject, including the immobilizing equipment, may be transferred to the resting device (i.e. the platform) of the magnetotactic system.

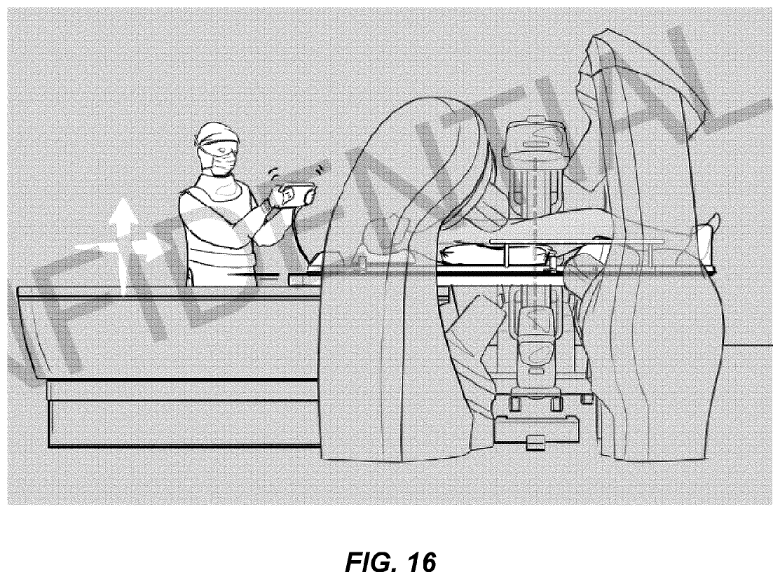

The position of the surface receiving the subject of the resting device of the magnetotactic system may be adjusted along one or more of the x, y and z axes (e.g. using a controller operated by a medical practitioner) to center the target zone (following the application of the isoshift) with respect to the isocenter of the magnetic heads of the magnetotactic system, as shown in FIG. 16. For instance, the height of the surface receiving the subject may be increased or decreased. The adjustment of the position of the surface receiving the subject may be performed based on the reference points located on the subject and information on the magnetic field generated by the magnetic heads of the magnetotactic system.

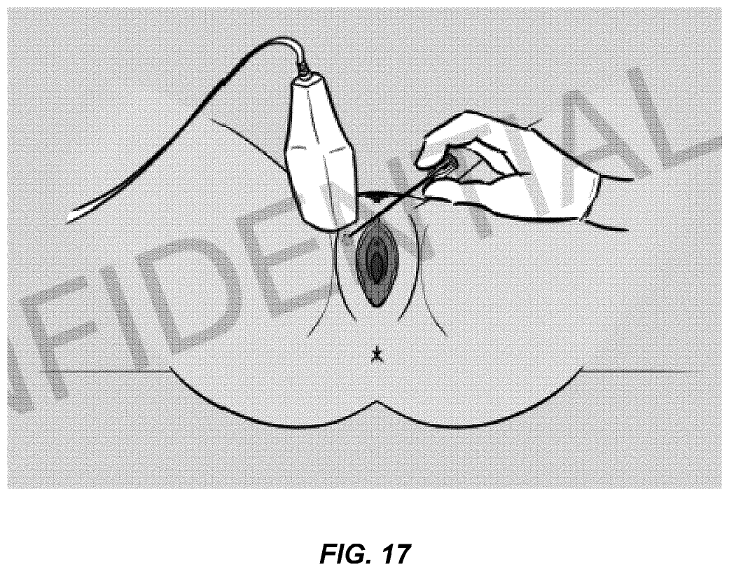

An introductory device (e.g. a needle, catheter with endoscope, etc.) may then be positioned on and/or inserted into the subject, at or next to the target zone, the introductory device for introducing the magneto-responsive entities into the subject (for instance, reference is made to FIG. 17, illustrating an exemplary tumor near the vulvar region).

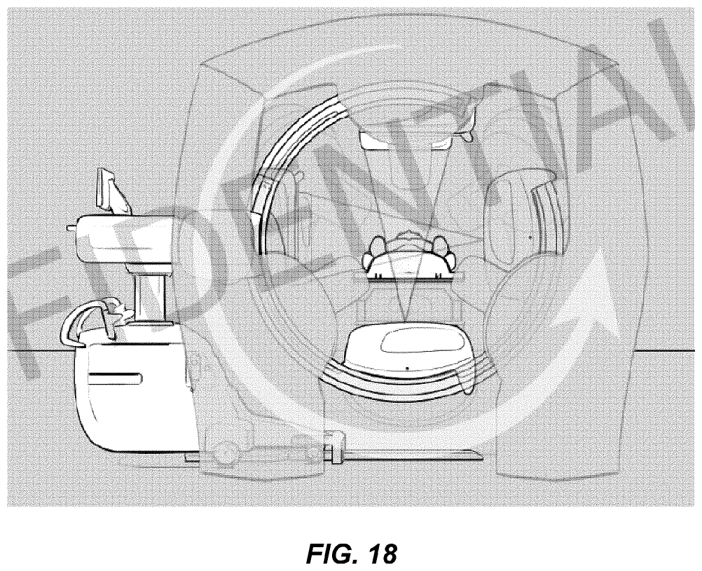

Further imagery may be performed on the subject (e.g. a cone-beam computed tomography (CBCT) scan performed using a C-arm) at step 2150 to visualize the position of the needle and/or the initial reference points (with added materials for imagery) and/or the treatment reference points (with added materials for imagery) with respect to the target zone, the position of the target zone further identifiable as a result of the treatment reference points added to the subject, as illustrated in FIG. 18. The imagery performed at step 2150 provides three-dimensional information on the subject.

In some examples, this additional imagery step may be compared to (mapped to and registered with) imagery (e.g. MRI imagery) taken previously of the tumor at step 2120, in order to confirm (to register) that the position of the tumor on the day of the medical intervention is that of tumor when the previous imagery was taken. This registration step may be used to pally physiological changes in the subject that could result in movement of the tumor (e.g. bowel movements, weight loss, weight gain, distended bladder, etc.) Moreover, this registration may enable validation of calculations performed previously to ascertain the location of target zone for the medical intervention.

In some examples, the position of the introductory device may be ascertained by injecting a contrast agent at the tip of the introductory device. The introductory device may then be withdrawn from the subject, the contrast agent indicating the location of the tip of the introductory device when performing imagery at step 2150. The visualization technique using a contrast agent may be useful for treatment of pancreatic cancer or rectal cancer.

The position of the subject may then be readjusted to account for the imagery information acquired at step 2150, at step 2160, resulting in a calibration of the position of magnetotactic system with respect to the subject and the target zone prior to the medical intervention. The repositioning may include moving the subject along any one of axes x, y and z, using, e.g., the controller for mobilizing the table of the magnetotactic system on which the subject is resting.

When repositioning of the subject is not possible or is not performed with respect to the magnetotactic system to align the target zone with the isocenter of the magnetotactic system, the difference between the isocenter of the magnetotactic system and the location of the target zone (as defined by the isoshift and the isocenter defined by the initial reference points) may be used to determine which magnetic field sequence(s) to apply when navigating the magneto-responsive entities from the injection point to the target zone. The adjustment or selection of magnetic field sequences accounts for this deviation of location of the target zone from the isocenter of the magnetotactic system. The deviation from the isocenter may be measured along one or more axes (x, y and z).

The difference in the selected magnetic field sequences depending on the location of the target zone from the isocenter enables the guidance of the magneto-responsive entities to the desired location in the subject.

It will be understood that the selection of the appropriate magnetic field sequences may depend on the structure of the magnetotactic system (e.g. the number of magnetic field sources, the position of the one or more magnetic field sources with respect to one another, the properties of the magnetic field sources, etc.) The determination of the appropriate magnetic field sequences depending on the location of the target zone with respect to the isocenter of the magnetotactic system may be performed through trial-and-error of the medical specialists operating the magnetotactic system, through the use of software, etc., depending on the properties and the complexity of the magnetotactic system.

The parameters of the magnetotactic system to generate the magnetic field may then be configured to generate a convergence point for the magneto-responsive entities such that they converge at the target zone, the subject now being positioned such that the target zone corresponds with the convergence point, i.e. isocenter, (e.g. aligns with) of the magnetic heads of the magnetotactic system.

A position of the introductory device (and of the point of introduction of the magneto-responsive entities into the subject) may be adjusted as a function of information obtained during imagery (e.g. a more optimal position for introducing the magneto-responsive entities into the subject, the presence of obstacles in the subject, etc.)

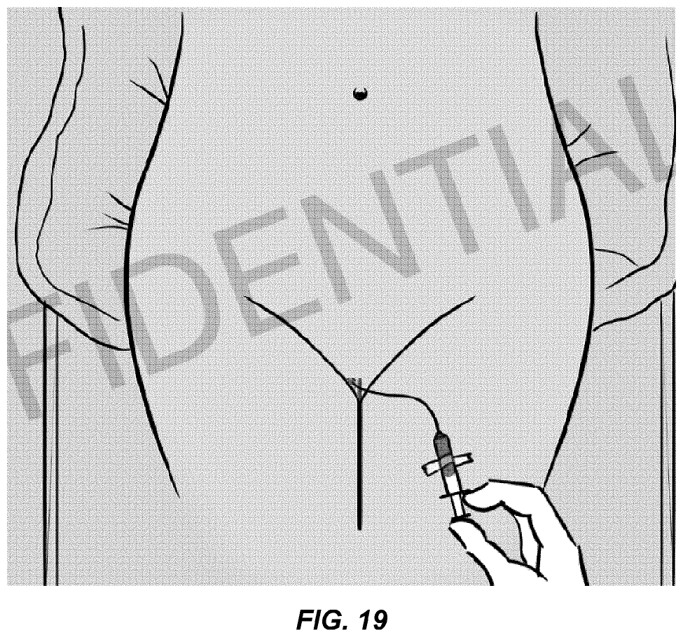

The magneto-responsive entities may then be introduced into the subject at, or next to, the target zone, as shown in FIG. 19, e.g., via injection, at step 2170, at the location confirmed via imagery.

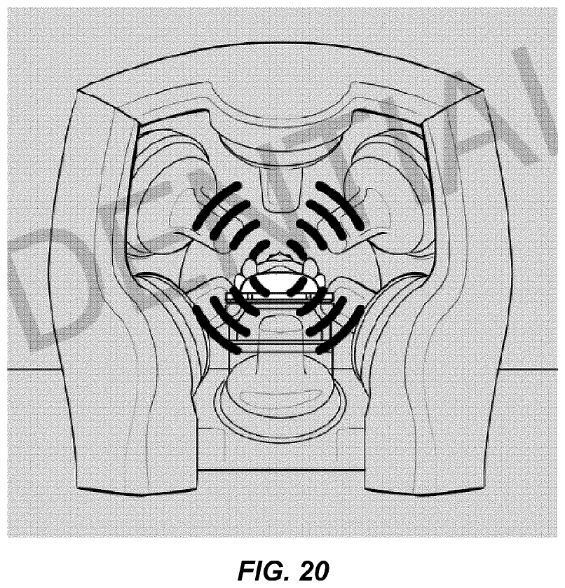

The parameters of the magnetic field generated by the magnetotactic system may then be controlled to generate a convergence point (e.g. a 3D convergence point), corresponding to the target zone through the positioning of the subject confirmed using the initial reference points and the corrected reference points, for navigating the magneto-responsive entities to the target zone at step 2180, as shown in FIG. 20.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is:

1. A method for preparing a subject for a medical intervention using magneto-responsive entities, tethered to at least one of a treatment agent, an imaging agent and a diagnostic agent, that responds to a magnetic field, the magneto-responsive entities to be steered in a body of the subject using the magnetic field, the magneto-responsive entities for delivering the at least one of the treatment agent, the imaging agent and the diagnostic agent in the subject, the method comprising:
defining reference points with respect to the subject indicative of a location of a target zone in the subject for one or more of treatment, diagnosis and imaging;
using the reference points to position the subject on a resting device in proximity to one or more sources for generating the magnetic field, the positioning performed in relation to the one or more sources;
performing imagery of the subject after the subject is positioned on the resting device; and
confirming an injection site;
whereby the magneto-responsive entities are to be introduced into the subject positioned on the table, and the magnetic field is applied to the magneto-responsive entities to obtain a response from the magneto-responsive entities using the magnetic field generated by the one or more sources to direct the magneto-responsive entities to the target zone.

2. The method as defined in claim 1, wherein the target zone is immobilized during the conducting of the medical intervention.

3. The method as defined in claim 2, wherein the target zone is immobilized using immobilizing equipment receiving the subject, and the method further comprising marking a position of the immobilized subject with respect to the immobilizing equipment.

4. The method as defined in any one of claims 1 to 3, further comprising:
defining initial reference points with respect to the subject for purposes of performing initial imagery of the subject, wherein the initial imagery identifies a location of the target zone; and
calculating an isoshift from an isocenter defined by the initial reference points and the location of the target zone, wherein the reference points are defined using the isoshift.

5. The method as defined in claim 4, wherein the initial imagery includes performing magnetic resonance imaging of the subject.

6. The method as defined in claim 4 or claim 5, wherein the initial imagery is registered with respect to the imagery of the subject after the subject is positioned on the table.

7. The method as defined in any one of claims 1 to 6, wherein the imagery is performed using a C-arm performing cone-beam computed tomography.

8. The method as defined in any one of claims 1 to 7, wherein the using the reference points to position the subject is performed with a laser system for positioning the reference points with respect to the one or more sources.

9. The method as defined in any one of claims 1 to 8, wherein the introducing is performed via injection using ultrasound to assist in positioning of a needle used for the injection.

10. The method as defined in any one of claims 1 to 9, wherein the reference points include tattoos applied to the subject.

11. The method as defined in any one of claims 1 to 9, wherein the reference points include one or more of fiducial markers, landmarks on the body of the subject, and the target zone.

12. The method as defined in any one of claims 1 to 11, wherein the target zone defines a tumor.

13. The method as defined in any one of claims 1 to 11, wherein the target zone corresponds to a hypoxic zone.

14. The method as defined in any one of claims 1 to 11, wherein the target zone is a necrotic zone.

15. The method as defined in claim 14, wherein the non-vascularized hypoxic zone is associated to an ischemic stroke, pulmonary hypertension, ischemic cardiopathy or diabetic retinopathy.

16. The method as defined in any one of claims 1 to 15, wherein the one or more sources include three pairs of magnetic heads, wherein each pair is aligned with regard to a distinct one of three axes including a x axis, a y axis and a z-axis.

17. The method as defined in any one of claims 1 to 16, wherein the imagery is performed after the injecting.

18. The method as defined in any one of claims 1 to 16, wherein the imagery is performed before the injecting.

19. The method as defined in any one of claims 1 to 18, further comprising applying markers.

20. A method for preparing a subject for a medical intervention on a subject using magneto-responsive entities, tethered to at least one of a treatment agent, an imaging agent and a diagnostic agent, that responds to a magnetic field generated by a plurality of magnetic field sources, the magneto-responsive entities for steering in a body of the subject using the magnetic field, the magneto-responsive entities for delivering the at least one of the treatment agent, the imaging agent and the diagnostic agent in the subject, the method comprising:
adjusting a position of the subject with respect to the isocenter of the plurality of magnetic field sources to at least improve alignment of a target zone of the subject with respect to the isocenter, the adjustment guided using reference points defined with respect to a body of the subject, the reference points being defined with respect to a body of the subject following an application of an isocenter shift calculated from a position of the target zone identified using imagery of the subject.
